SEQUENCE LISTING (57 sequences)

SEQ ID NO: 1 - DNA, 84 bp, Artificial Sequence
  Description of Artificial SequencePrimer C
gaaattaatacgactcactatagggagaccacaacggtttccctctagaaataattttgtttaactttaagaaggagatatacc

SEQ ID NO: 2 - DNA, 71 bp, Artificial Sequence
  Description of Artificial SequencePrimer D
caaaaaacccctcaagacccgtttagaggccccaaggggggccgccagtgtgctgaattcgccttttatta

SEQ ID NO: 3 - DNA, 30 bp, Artificial Sequence
  Description of Artificial SequencePrimer A without hairpinloop
aggagatataccatgactagcaaaggagaa

SEQ ID NO: 4 - DNA, 42 bp, Artificial Sequence
  Description of Artificial SequencePrimer A Stem Length 4 bp
aggagatataccatgactaattttagtactagcaaaggagaa

SEQ ID NO: 5 - DNA, 45 bp, Artificial Sequence
  Description of Artificial SequencePrimer A Stem Length 5 bp
aggagatataccatgactgtttatacagtaactagcaaaggagaa

SEQ ID NO: 6 - DNA, 48 bp, Artificial Sequence
  Description of Artificial SequencePrimer A Stem Length 6 bp
aggagatataccatgactggtcaattaccagtaactagcaaaggagaa

SEQ ID NO: 7 - DNA, 51 bp, Artificial Sequence
  Description of Artificial SequencePrimer A Stem Length 7 bp
aggagatataccatgactgctttacatcaagcagtaactagcaaaggagaa

SEQ ID NO: 8 - DNA, 51 bp, Artificial Sequence
  Description of Artificial SequencePrimer A Stem Length 8 bp
aggagatataccatgactgcacgtgatcgtgcagtaactagcaaaggagaa

SEQ ID NO: 9 - DNA, 30 bp, Artificial Sequence
  Description of Artificial SequencePrimer B
attcgccttttattaatgatgatgatgatg

SEQ ID NO: 10 - DNA, 60 bp, Artificial Sequence
  Description of Artificial SequencePrimer A
aggagatataccatgactagcactgcacgtgcatcgtgcagtgtaaaaggagaagaactt

SEQ ID NO: 11 - DNA, 63 bp, Artificial Sequence
  Description of Artificial SequencePrimer A
aggagatataccatgactagcaaaactgcacgtgcatcgtgcagtgtaggagaagaacttttc

SEQ ID NO: 12 - DNA, 66 bp, Artificial Sequence
  Description of Artificial SequencePrimer A
aggagatataccatgactagcaaaggaactgcacgtgcatcgtgcagtgtagaagaacttttcact

SEQ ID NO: 13 - DNA, 69 bp, Artificial Sequence
  Description of Artificial SequencePrimer A
aggagatataccatgactagcaaaggagaaactgcacgtgcatcgtgcagtgtagaacttttcactgga

SEQ ID NO: 14 - DNA, 72 bp, Artificial Sequence
  Description of Artificial SequencePrimer A
aggagatataccatgactagcaaaggagaagaaactgcacgtgcatcgtgcagtgtacttttcactggagtt

SEQ ID NO: 15 - DNA, 75 bp, Artificial Sequence
  Description of Artificial SequencePrimer A
aggagatataccatgactagcaaaggagaagaacttactgcacgtgcatcgtgcagtgtattcactggagttgtc

SEQ ID NO: 16 - DNA, 71 bp, Artificial Sequence
  Description of Artificial SequencePrimer D
caaaaaacccctcaagacccgtttagaggccccaaggggttgggagtagaatgttaaggattagtttatta

SEQ ID NO: 17 - DNA, 60 bp, Artificial Sequence
  Description of Artificial SequencePrimer A Variant
aggagatataccatgaaatatacatattctctgcacgtgatcgtgcaggctaacaccgcg

SEQ ID NO: 18 - DNA, 60 bp, Artificial Sequence
  Description of Artificial SequencePrimer A Variant
aggagatataccatgaaaacatattattctctgcacgtgatcgtgcaggctaacaccgcg

SEQ ID NO: 19 - DNA, 60 bp, Artificial Sequence
  Description of Artificial SequencePrimer A Variant
aggagatataccatgaaatattcttatacactgcacgtgatcgtgcaggctaacaccgcg

SEQ ID NO: 20 - DNA, 60 bp, Artificial Sequence
  Description of Artificial SequencePrimer A Variant
aggagatataccatgaaatattattctacactgcacgtgatcgtgcaggctaacaccgcg

SEQ ID NO: 21 - DNA, 60 bp, Artificial Sequence
  Description of Artificial SequencePrimer A Variant
aggagatataccatgaaatatacatattcactgcacgtgatcgtgcaggctaacaccgcg

SEQ ID NO: 22 - DNA, 60 bp, Artificial Sequence
  Description of Artificial SequencePrimer A Variant
aggagatataccatgaaaacatattattcactgcacgtgatcgtgcaggctaacaccgcg

SEQ ID NO: 23 - DNA, 60 bp, Artificial Sequence
  Description of Artificial SequencePrimer A Variant
aggagatataccatgaaatattcatatacactgcacgtgatcgtgcaggctaacaccgcg

SEQ ID NO: 24 - DNA, 60 bp, Artificial Sequence
  Description of Artificial SequencePrimer A Variant
aggagatataccatgaaatattattcaacactgcacgtgatcgtgcaggctaacaccgcg

SEQ ID NO: 25 - DNA, 60 bp, Artificial Sequence
  Description of Artificial SequencePrimer A Variant
aggagatataccatgcatcatcatcatcatctgcacgtgatcgtgcaggctaacaccgcg

SEQ ID NO: 26 - DNA, 27 bp, Artificial Sequence
  Description of Artificial SequencePrimer Wildtype
aggagatataccatggctaacaccgcg

SEQ ID NO: 27 - DNA, 48 bp, Artificial Sequence
  Description of Artificial SequencePrimer B
aggattagtttattaatgatgatgatgatgatggcgccgggtgcgcga

SEQ ID NO: 28 - DNA, 60 bp, Artificial Sequence
  Description of Artificial SequencePrimer A Variant
aggagatataccatgaaatatacatattctctgcacgtgatcgtgcagggtgccccgacg

SEQ ID NO: 29 - DNA, 60 bp, Artificial Sequence
  Description of Artificial SequencePrimer A Variant
aggagatataccatgaaaacatattattctctgcacgtgatcgtgcagggtgccccgacg

SEQ ID NO: 30 - DNA, 60 bp, Artificial Sequence
  Description of Artificial SequencePrimer A Variant
aggagatataccatgaaatattcttatacactgcacgtgatcgtgcagggtgccccgacg

SEQ ID NO: 31 - DNA, 60 bp, Artificial Sequence
  Description of Artificial SequencePrimer A Variant
aggagatataccatgaaatattattctacactgcacgtgatcgtgcagggtgccccgacg

SEQ ID NO: 32 - DNA, 60 bp, Artificial Sequence
  Description of Artificial SequencePrimer A Variant
aggagatataccatgaaatatacatattcactgcacgtgatcgtgcagggtgccccgacg

SEQ ID NO: 33 - DNA, 60 bp, Artificial Sequence
  Description of Artificial SequencePrimer A Variant
aggagatataccatgaaaacatattattcactgcacgtgatcgtgcagggtgccccgacg

SEQ ID NO: 34 - DNA, 60 bp, Artificial Sequence
  Description of Artificial SequencePrimer A Variant
aggagatataccatgaaatattcatatacactgcacgtgatcgtgcagggtgccccgacg

SEQ ID NO: 35 - DNA, 60 bp, Artificial Sequence
  Description of Artificial SequencePrimer A Variant
aggagatataccatgaaatattattcaacactgcacgtgatcgtgcagggtgccccgacg

SEQ ID NO: 36 - DNA, 60 bp, Artificial Sequence
  Description of Artificial SequencePrimer A Variant
aggagatataccatgcatcatcatcatcatctgcacgtgatcgtgcagggtgccccgacg

SEQ ID NO: 37 - DNA, 27 bp, Artificial Sequence
  Description of Artificial SequencePrimer A Wildtype
aggagatataccatgggtgccccgacg

SEQ ID NO: 38 - DNA, 49 bp, Artificial Sequence
  Description of Artificial SequencePrimer B
aggattagtttattaatgatgatgatgatgatgatccatggcagccagc

SEQ ID NO: 39 - DNA, 60 bp, Artificial Sequence
  Description of Artificial SequencePrimer
aggagatataccatgaaatatacatattctctgcacgtgatcgtgcaggagttggggccc

SEQ ID NO: 40 - DNA, 60 bp, Artificial Sequence
  Description of Artificial SequencePrimer
aggagatataccatgaaaacatattattctctgcacgtgatcgtgcaggagttggggccc

SEQ ID NO: 41 - DNA, 60 bp, Artificial Sequence
  Description of Artificial SequencePrimer
aggagatataccatgaaatattcttatacactgcacgtgatcgtgcaggagttggggccc

SEQ ID NO: 42 - DNA, 60 bp, Artificial Sequence
  Description of Artificial SequencePrimer
aggagatataccatgaaatattattctacactgcacgtgatcgtgcaggagttggggccc

SEQ ID NO: 43 - DNA, 60 bp, Artificial Sequence
  Description of Artificial SequencePrimer
aggagatataccatgaaatatacatattcactgcacgtgatcgtgcaggagttggggccc

SEQ ID NO: 44 - DNA, 60 bp, Artificial Sequence
  Description of Artificial SequencePrimer
aggagatataccatgaaaacatattattcactgcacgtgatcgtgcaggagttggggccc

SEQ ID NO: 45 - DNA, 60 bp, Artificial Sequence
  Description of Artificial SequencePrimer
aggagatataccatgaaatattcatatacactgcacgtgatcgtgcaggagttggggccc

SEQ ID NO: 46 - DNA, 60 bp, Artificial Sequence
  Description of Artificial SequencePrimer
aggagatataccatgaaatattattcaacactgcacgtgatcgtgcaggagttggggccc

SEQ ID NO: 47 - DNA, 60 bp, Artificial Sequence
  Description of Artificial SequencePrimer
aggagatataccatgcatcatcatcatcatctgcacgtgatcgtgcaggagttggggccc

SEQ ID NO: 48 - DNA, 27 bp, Artificial Sequence
  Description of Artificial SequencePrimer A Wildtype
aggagatataccatggagttggggccc

SEQ ID NO: 49 - DNA, 45 bp, Artificial Sequence
  Description of Artificial SequencePrimer B
aggattagtttattattaatgatgatgatgatgatgagaaccccc

SEQ ID NO: 50 - DNA, 431 bp, Artificial Sequence
  Description of Artificial Sequence Expression construct for mutant 1
gaaattaatacgactcactatagggagaccacaacggtttccctctagaaataattttgtttaactttaagaaggagatataccatgaaatatacatattctctgcacgtgatcgtgcaggctaacaccgcgccgggacccacggtggccaacaagcgggacgaaaaacaccgtcacgtcgttaacgtcgttttggagctgccgaccgagatatcagaggccacccacccggtgttggccaccatgctgagcaagtacacgcgcatgtccagcctgtttaatgacaagtgcgcctttaagctggacctgttgcgcatggtagccgtgtcgcgcacccggcgccatcatcatcatcatcattaataaactaatccttaacattctactcccaaccccttggggcctctaaacgggtcttgaggggttttttg

SEQ ID NO: 51 - DNA, 398 bp, Artificial Sequence
  Description of Artificial Sequence Expression construct for wildtype
gaaattaatacgactcactatagggagaccacaacggtttccctctagaaataattttgtttaactttaagaaggagatataccatggctaacaccgcgccgggacccacggtggccaacaagcgggacgaaaaacaccgtcacgtcgttaacgtcgttttggagctgccgaccgagatatcagaggccacccacccggtgttggccaccatgctgagcaagtacacgcgcatgtccagcctgtttaatgacaagtgcgcctttaagctggacctgttgcgcatggtagccgtgtcgcgcacccggcgccatcatcatcatcatcattaataaactaatccttaacattctactcccaaccccttggggcctctaaacgggtcttgaggggttttttg

SEQ ID NO: 52 - DNA, 632 bp, Artificial Sequence
  Description of Artificial Sequence Expresion construct for mutant 1
gaaattaatacgactcactatagggagaccacaacggtttccctctagaaataattttgtttaactttaagaaggagatataccatgaaatatacatattctctgcacgtgatcgtgcagggtgccccgacgttgccccctgcctggcagccctttctcaaggaccaccgcatctctacattcaagaactggcccttcttggagggctgcgcctgcaccccggagcggatggccgaggctggcttcatccactgccccactgagaacgagccagacttggcccagtgtttcttctgcttcaaggagctggaaggctgggagccagatgacgaccccatagaggaacataaaaagcattcgtccggttgcgctttcctttctgtcaagaagcagtttgaagaattaacccttggtgaatttttgaaactggacagagaaagagccaagaacaaaattgcaaaggaaaccaacaataagaagaaagaatttgaggaaactgcgaagaaagtgcgccgtgccatcgagcagctggctgccatggatcatcatcatcatcatcattaataaactaatccttaacattctactcccaaccccttggggcctctaaacgggtcttgaggggttttttg

SEQ ID NO: 53 - DNA, 599 bp, Artificial Sequence
  Description of Artificial Sequence Expression construct for Wildtype
gaaattaatacgactcactatagggagaccacaacggtttccctctagaaataattttgtttaactttaagaaggagatataccatgggtgccccgacgttgccccctgcctggcagccctttctcaaggaccaccgcatctctacattcaagaactggcccttcttggagggctgcgcctgcaccccggagcggatggccgaggctggcttcatccactgccccactgagaacgagccagacttggcccagtgtttcttctgcttcaaggagctggaaggctgggagccagatgacgaccccatagaggaacataaaaagcattcgtccggttgcgctttcctttctgtcaagaagcagtttgaagaattaacccttggtgaatttttgaaactggacagagaaagagccaagaacaaaattgcaaaggaaaccaacaataagaagaaagaatttgaggaaactgcgaagaaagtgcgccgtgccatcgagcagctggctgccatggatcatcatcatcatcatcattaataaactaatccttaacattctactcccaaccccttggggcctctaaacgggtcttgaggggttttttg

SEQ ID NO: 54 - DNA, 1400 bp, Artificial Sequence
  Description of Artificial Sequence Expression construct for mutant 1
gaaattaatacgactcactatagggagaccacaacggtttccctctagaaataattttgtttaactttaagaaggagatataccatgaaatatacatattctctgcacgtgatcgtgcaggagttggggcccctagaaggtggctacctggagcttcttaacagcgatgctgaccccctgtgcctctaccacttctatgaccagatggacctggctggagaagaagagattgagctctactcagaacccgacacagacaccatcaactgcgaccagttcagcaggctgttgtgtgacatggaaggtgatgaagagaccagggaggcttatgccaatatcgcggaactggaccagtatgtcttccaggactcccagctggagggcctgagcaaggacattttcaagcacataggaccagatgaagtgatcggtgagagtatggagatgccagcagaagttgggcagaaaagtcagaaaagacccttcccagaggagcttccggcagacctgaagcactggaagccagctgagccccccactgtggtgactggcagtctcctagtgggaccagtgagcgactgctccaccctgccctgcctgccactgcctgcgctgttcaaccaggagccagcctccggccagatgcgcctggagaaaaccgaccagattcccatgcctttctccagttcctcgttgagctgcctgaatctccctgagggacccatccagtttgtccccaccatctccactctgccccatgggctctggcaaatctctgaggctggaacaggggtctccagtatattcatctaccatggtgaggtgccccaggccagccaagtaccccctcccagtggattcactgtccacggcctcccaacatctccagaccggccaggctccaccagccccttcgctccatcagccactgacctgcccagcatgcctgaacctgccctgacctcccgagcaaacatgacagagcacaagacgtcccccacccaatgcccggcagctggagaggtctccaacaagcttccaaaatggcctgagccggtggagcagttctaccgctcactgcaggacacgtatggtgccgagcccgcaggcccggatggcatcctagtggaggtggatctggtgcaggccaggctggagaggagcagcagcaagagcctggagcgggaactggccaccccggactgggcagaacggcagctggcccaaggaggcctggctgaggtgctgttggctgccaaggagcaccggcggccgcgtcgactcgagcgagctcccggggggggttctcatcatcatcatcatcattaataataaactaatccttaacattctactcccaaccccttggggcctctaaacgggtcttgaggggttttttg

SEQ ID NO: 55 - DNA, 1367 bp, Artificial Sequence
  Description of Artificial Sequence Expression construct for wildtype
gaaattaatacgactcactatagggagaccacaacggtttccctctagaaataattttgtttaactttaagaaggagatataccatggagttggggcccctagaaggtggctacctggagcttcttaacagcgatgctgaccccctgtgcctctaccacttctatgaccagatggacctggctggagaagaagagattgagctctactcagaacccgacacagacaccatcaactgcgaccagttcagcaggctgttgtgtgacatggaaggtgatgaagagaccagggaggcttatgccaatatcgcggaactggaccagtatgtcttccaggactcccagctggagggcctgagcaaggacattttcaagcacataggaccagatgaagtgatcggtgagagtatggagatgccagcagaagttgggcagaaaagtcagaaaagacccttcccagaggagcttccggcagacctgaagcactggaagccagctgagccccccactgtggtgactggcagtctcctagtgggaccagtgagcgactgctccaccctgccctgcctgccactgcctgcgctgttcaaccaggagccagcctccggccagatgcgcctggagaaaaccgaccagattcccatgcctttctccagttcctcgttgagctgcctgaatctccctgagggacccatccagtttgtccccaccatctccactctgccccatgggctctggcaaatctctgaggctggaacaggggtctccagtatattcatctaccatggtgaggtgccccaggccagccaagtaccccctcccagtggattcactgtccacggcctcccaacatctccagaccggccaggctccaccagccccttcgctccatcagccactgacctgcccagcatgcctgaacctgccctgacctcccgagcaaacatgacagagcacaagacgtcccccacccaatgcccggcagctggagaggtctccaacaagcttccaaaatggcctgagccggtggagcagttctaccgctcactgcaggacacgtatggtgccgagcccgcaggcccggatggcatcctagtggaggtggatctggtgcaggccaggctggagaggagcagcagcaagagcctggagcgggaactggccaccccggactgggcagaacggcagctggcccaaggaggcctggctgaggtgctgttggctgccaaggagcaccggcggccgcgtcgactcgagcgagctcccggggggggttctcatcatcatcatcatcattaataataaactaatccttaacattctactcccaaccccttggggcctctaaacgggtcttgaggggttttttg

SEQ ID NO: 56 - DNA, 938 bp, Artificial Sequence
  Description of Artificial SequenceExpression construct
gaaattaatacgactcactatagggagaccacaacggtttccctctagaaataattttgtttaactttaagaaggagatataccatgaaatatacatattctctgcacgtgatcgtgcagactagcaaaggagaagaacttttcactggagttgtcccaattcttgttgaattagatggtgatgttaatgggcacaaattttctgtcagtggagagggtgaaggtgatgctacatacggaaagcttacccttaaatttatttgcactactggaaaactacctgttccatggccaacacttgtcactactttctcttatggtgttcaatgcttttcccgttatccggatcatatgaaacggcatgactttttcaagagtgccatgcccgaaggttatgtacaggaacgcactatatctttcaaagatgacgggaactacaagacgcgtgctgaagtcaagtttgaaggtgatacccttgttaatcgtatcgagttaaaaggtattgattttaaagaagatggaaacattctcggacacaaactcgagtacaactataactcacacaatgtatacatcacggcagacaaacaaaagaatggaatcaaagctaacttcaaaattcgccacaacattgaagatggatccgttcaactagcagaccattatcaacaaaatactccaattggcgatggccctgtccttttaccagacaaccattacctgtcgacacaatctgccctttcgaaagatcccaacgaaaagagagaccacatggtccttcttgagtttgtaacagctgctgggattacacatggcatggatgaactatacaaacccggggggggttctcatcatcatcatcatcattaataaactaatccttaacattctactcccaaccccttggggcctctaaacgggtcttgaggggttttttg

SEQ ID NO: 57 - DNA, 905 bp, Artificial Sequence
  Description of Artificial Sequence Expression construct
gaaattaatacgactcactatagggagaccacaacggtttccctctagaaataattttgtttaactttaagaaggagatataccatgactagcaaaggagaagaacttttcactggagttgtcccaattcttgttgaattagatggtgatgttaatgggcacaaattttctgtcagtggagagggtgaaggtgatgctacatacggaaagcttacccttaaatttatttgcactactggaaaactacctgttccatggccaacacttgtcactactttctcttatggtgttcaatgcttttcccgttatccggatcatatgaaacggcatgactttttcaagagtgccatgcccgaaggttatgtacaggaacgcactatatctttcaaagatgacgggaactacaagacgcgtgctgaagtcaagtttgaaggtgatacccttgttaatcgtatcgagttaaaaggtattgattttaaagaagatggaaacattctcggacacaaactcgagtacaactataactcacacaatgtatacatcacggcagacaaacaaaagaatggaatcaaagctaacttcaaaattcgccacaacattgaagatggatccgttcaactagcagaccattatcaacaaaatactccaattggcgatggccctgtccttttaccagacaaccattacctgtcgacacaatctgccctttcgaaagatcccaacgaaaagagagaccacatggtccttcttgagtttgtaacagctgctgggattacacatggcatggatgaactatacaaacccggggggggttctcatcatcatcatcatcattaataaactaatccttaacattctactcccaaccccttggggcctctaaacgggtcttgaggggttttttg

(19) United States
(12) Patent Application Publication
(10) Pub. No.: US 2006/0264612 A1
(43) Pub. Date: Nov. 23, 2006

(54) Optimised protein synthesis

(76) Inventors: Manfred Watzele, Weilheim (DE); Bernd Buchberger, Peissenberg (DE); Michael Paulus, Augsburg (DE)

Correspondence Address: BARNES & THORNBURG LLP, 11 SOUTH MERIDAN STREET, INDIANAPOLIS, IN, 46204, US

(21) Appl. No.: 10/538,405

(22) PCT Filed: Dec. 9, 2003
(86) PCT No.: PCT/EP03/13964

§ 371 Date: Jun. 9, 2005

(30) Foreign Application Priority Data
Dec. 9, 2002 (DE) 10257479.0

(51) Int. Cl. C07K 14/705; C12N 1/21; C12N 1/18; C12P 21/06; C12N 5/06; C12N 5/08

(52) U.S. Cl. 530/350; 435/69.1; 435/252.3; 435/320.1; 435/254.2; 435/366; 435/348; 435/252.31

(57) ABSTRACT

The invention concerns a method for the optimized production of proteins in an in vitro or in vivo expression system and reagents suitable therefor.

The invention concerns a method for the optimized production of proteins in an in vitro or in vivo expression system and reagents suitable therefor.

Hannig, G. & Makrides, S. C. (1998) Tibtech Vol 16, pp 54-60 have described strategies for optimizing heterologous protein expression in E. coli. A key factor in this connection is the efficiency of the initiation of translation in which the usage of particular codons plays a certain role. Thus George et al. (1985) DNA Vol 4, pp 273-281, show that the expression of a heterologous gene can be increased by using codons in the region after the start codon that are frequently utilized in E. coli genes. It is predominantly structural elements at the 5′ end of mRNA that are particularly important for translation initiation. Makrides (1996) Microbiol. Rev. Vol 60, pp 512-538 described various translation enhancer sequences such as the sequence from the T7 phage gene 10 leader and a U-rich sequence from the 5′-untranslated region of some mRNAs such as the atpE gene of E. coli.

No translation initiation sequences have been described up to now which can be used universally. However, strategies have been described that reduce the potential for the formation of secondary structures at the 5′-end of the mRNA. In particular the ribosomal binding site was enriched with adenine and thymine building blocks. Stenstöm et al. (2001) Gene Vol. 263, pp 273-284 showed that strongly expressed E. coli genes have a high content of adenines especially in the +2 codon following the start codon. However, there are also many positive and negative exceptions to this rule.

Finally Pederson-Lane et al. (1997) Protein Expr. Purif. Vol. 10, pp 256-262, showed that a high GC content directly after the start codon has a negative effect on expression and that the expression of thymidilate synthase could be increased to 25% of the total protein by converting the purine bases of the third, fourth and fifth codon into thymidine bases.

It is assumed that the ability of the 30 S ribosome subunit to gain access to the messenger RNA plays an important role in all of these measures. It is particularly important that there is free contact with the sequence named after Shine and Dalgarno directly in front of the start codon and contact with the start codon itself. If, however, these sequence elements are bound in stable RNA secondary structures, the initiation of translation progresses very inefficiently. Tessier et al. (1984) Nucl. Ac. Res. Vol 12, pp 7663-7675 showed in a systematic investigation that this form of secondary structures that resemble stems and loops (so-called stem-loops or hairpin-loops) can be broken by a targeted mutation thus considerably increasing the efficiency of translation. The effect of these secondary structures on translation can be calculated on the basis of their thermodynamic parameters. Thus a stabilization of 1.4 kcal/mol results in a 10-fold reduction in expression (Gold (1988) Ann. Rev. Biochem., Vol 57, pp 199-233) and a stabilization of 2.3 kcal/mol reduces the binding of the ribosome by an order of magnitude (deSmit & van Duin (1994), J. Mol. Biol. Vol 244, pp 144-150).

The so-called “downstream box” which is a sequence element directly after the start codon of the T7 genes with homology to the ribosomal 16S RNA has been described by Sprengart et al. (1996) EMBO Vol 15, pp 665-674 as another translation enhancer. It is assumed that this element increases the binding of the 30S ribosomal subunit by an interaction of the two homologous base pairs. However, this element is also not suitable as a universal translation enhancer.

The disadvantages of the known processes are that an optimization of the 5′ region of the mRNA either in the 5′-untranslated region or in the translated region has to be carried out for each new gene in order to optimize the codon usage or to avoid undesired secondary structures of the mRNA that have an effect on the Shine-Dalgarno sequence or the start codon. This usually requires a laborious analysis of the RNA structure with appropriate programs (e.g. Mukund et al. (1999) Curr. Science Vol 76, pp 1486-1490, or Jaeger et al. (1990) Meth. Enzymol. Vol 183, pp 281-306) as well as several PCR amplifications and cloning steps. If one intends to express a large number of genes for example from a gene bank in this manner, then the sequence has to be exactly known in each case which is why these methods cannot be used for unknown genes. Even if the sequences were known, this method would be much more laborious than a universally applicable method.

Another approach for enhancing translation is to form a fusion protein with a strongly expressed gene as a universal translation enhancer on the C-terminal end of which the desired gene is placed. An example of the success of this strategy is the fusion with the ubiquitin gene that was carried out by Butt et al. (1989) PNAS Vol 86, pp 2540-2544.

However, even this approach cannot be easily applied to the expression of any genes. If, for example, fusion proteins are used then a fusion of a greater or lesser size is attached to the N-terminus of the protein which due to the size and properties of the fusion partner can interfere with the function of the desired protein. The smaller the size that is selected for the fusion proteins or parts thereof, the lower is their translation-enhancing effect in many cases. Large fusion proteins exhibit a further disadvantage in prokaryotic expression systems: There is a concurrent increase in the probability of incomplete transcription or translation by premature termination or internal initialization. Also the probability of proteolytic degradation is increased.

Hence there is a need to provide a method for the optimized production of proteins in which the disadvantages of the prior art are at least partially eliminated.

A subject matter of the invention is a method for producing a protein comprising the steps:
(a) providing a nucleic acid sequence coding for the protein in which a heterologous nucleic acid sequence is inserted on the 3′ side of the translation start codon in the correct reading frame, said nucleic acid being selected such that a stem-loop structure is formed on the 3′ side of the translation start codon at a distance of 6-30 nucleotides,
(b) providing an expression system suitable for expressing the protein and
(c) introducing the nucleic acid sequence according to (a) into the expression system according to (b) under such conditions that the protein is synthesized.

The solution according to the invention for a universally optimized expression construct comprises the insertion of a small heterologous DNA sequence element having preferably a maximum of 201 base pairs, particularly preferably a maximum of 45 base pairs, directly after the start codon of the gene to be expressed, which substantially prevents the formation of stable stem-loop structures in the region of the Shine-Dalgarno sequence and of the start codon and thus results in an optimized translation initiation and optimized protein synthesis. Hence a fusion protein is formed in which preferably only a small peptide having a maximum of 67 amino acids and particularly preferably a maximum of 15 amino acids is attached to the desired protein.

An important prerequisite for the heterologous DNA sequence element is that it is inserted in the correct reading frame i.e. that the frame is not shifted in the gene to be expressed. Another important property of the heterologous DNA sequence element is that a stable stem-loop structure can form in the transcribed RNA at a distance of 6-30 bases, preferably 12-21 bases behind the start codon where the base pairing in the stem-loop structure is at least partially effected by the inserted sequence. This stem-loop structure should be such that it can be opened again by the ribosome after translation has been initiated and thus does not result in a termination of translation. This stem-loop structure that is formed by inserting the heterologous nucleic acid sequence into the expression construct can form in the same manner in almost any gene and thus prevent sequences that are important for translation initiation that are in front of the loop from forming large secondary structures with the coding sequence of the gene. The region directly in front of this stem-loop structure and after the start codon is preferably a sequence without a secondary structure and which can also not form a secondary structure with the 5′-untranslated region. A sequence which has a low content of GC is particularly preferred in this region since such a sequence reduces the formation of stable secondary structures with sequences within the translated region.

The heterologous nucleic acid sequence element can be inserted into the target sequence e.g. into a plasmid vector for expressing heterologous genes by using known cloning or/and amplification techniques. It is for example possible to construct this sequence by PCR primers for cloning the desired gene or by primers which can be used to produced DNA expression constructs for in vitro protein expression.

The method according to the invention can be used to produce and optionally isolate proteins in in vitro expression systems. Examples of suitable in vitro expression systems are prokaryotic in vitro expression systems such as lysates of gram-negative bacteria for example of Escherichia coli, or gram-positive bacteria for example Bacillus subtilis or eukaryotic in vitro expression systems such as lysates of mammalian cells, for example of rabbits, reticulocytes, human tumour cell lines, hamster cell lines or other vertebrate cells such as oocytes and eggs of fish and amphibia, as well as insect cell lines, yeast cells, algal cells or extracts of plant seeds.

Alternatively the protein can be produced in an in vivo expression system in which case it is possible to use a prokaryotic cell e.g. a gram-negative prokaryotic host cell in particular an E. coli cell or a gram-positive prokaryotic cell in particular a Bacillus subtilis cell, a eukaryotic host cell e.g. a yeast cell, an insect cell or a vertebrate cell in particular an amphibian, fish, bird or mammalian cell or a non-human eukaryotic host organism as the expression system.

The heterologous nucleic acid sequence can be introduced into the nucleic acid coding for the desired protein by standard methods of molecular biology e.g. by cloning such as restriction cleavage or/and ligation, by recombination or/and by nucleic acid amplification. The nucleic acid target sequence can be present on a suitable vector e.g. a plasmid vector for the expression of heterologous genes or on a construct for an in vitro protein expression. The nucleic acid amplification is particularly preferably carried out in one or more steps in which the heterologous nucleic acid sequence and optionally expression control sequences such promoters, ribosomal binding sites and terminators can be attached to the nucleic acid sequence coding for the desired protein by selecting suitable primers. A two-step PCR is particularly preferred where in a first step at least a part of the heterologous nucleic acid sequence is attached to a nucleic acid target sequence which codes for the desired protein and expression control sequences are attached in a second step. A preferred embodiment for carrying out a two-step PCR is illustrated in the examples.

The heterologous nucleic acid sequence which is able to form a stem-loop structure on the 3′ side of the translation start codon is inserted into the nucleic acid sequence coding for the desired protein in the correct reading frame on the 3′ side of the translation start codon which is usually the first ATG codon. It is preferably inserted at a distance of up to 6 nucleotides and particularly preferably directly after the translation start codon. In this connection an insertion in the “correct reading frame” means that there is no shift in the reading frame in the protein-coding nucleic acid sequence. This in turn means that the length of the heterologous nucleic acid sequence measured in nucleotides is a multiple of 3. Its length is preferable in the range of 6-201 nucleotides, particularly preferably in the range of 12-45 nucleotides.

The heterologous nucleic acid sequence is inserted into the protein-coding nucleic acid sequence such that a stem-loop structure is formed at a suitable distance on the 3′ side of the translation codon. The distance (between the last nucleotide of the translation start codon and the first nucleotide of the stem) is advantageously 6-30 nucleotides, particularly preferably 12-21 nucleotides. The heterologous nucleic acid sequence preferably contains an AT-rich region on the 5′ side of the sequences that are provided for the formation of the stem-loop structure i.e. a region having an AT content of >50%, in particular >60%.

The length of the stem in the stem-loop structure is preferably in the range of 4 to 12 nucleotides, particularly preferably 5 to 10 nucleotides. The stem of the stem-loop structure preferably contains two sections that are completely complementary to one another. However, one or more base mismatches may also be present provided they do not greatly reduce the stability. The base pairs in the stem can be AT and GC base pairs and combinations thereof. It is preferable to have a proportion of GC base pairs of >50%. The length of the loop is preferably 2 to 8 nucleotides but it is not particularly critical. The thermodynamic stability of the stem-loop structure is expediently high enough to prevent the formation of a secondary structure in the region of the ATG start codon, of the 15 nucleotides on the 5′ side which comprise the Shine-Dalgarno sequence and at least of the 5 nucleotides on the 3′ side. On the other hand the thermodynamic stability of the stem-loop structure should not be of such a magnitude that it impedes the processing of the ribosome on the mRNA. The thermodynamic stability of the stem-loop structure is preferably in the range of −4 to −15 kcal/mol.

The expression control sequences used to express the desired protein comprise promoters, ribosomal binding sites i.e. Shine-Dalgarno sequences for prokaryotic expression systems or Kozak sequences for eukaryotic expression systems, enhancers, terminators, polyadenylation sequences etc. A person skilled in the art knows such expression control sequences from standard textbooks of molecular biology e.g. Sambrook et al. (1989) Molecular Cloning, A Laboratory Manual, Cold Spring Harbor or Ausubel et al. (1989) Current Protocols in Molecular Biology, John Wiley & Sons, New York.

Furthermore, the heterologous nucleic acid sequence can also contain sections which code for a purification domain e.g. a poly-His domain, a FLAG epitope domain etc. or/and a proteinase-recognition domain e.g. an IgA protease or factor X domain. The purification domain can simplify the isolation of the desired protein e.g. from an in vitro translation preparation or a host cell or the medium used for culturing. The heterologous peptide sequence can be cleaved from the desired protein by protease cleavage within the protease recognition domain.

The heterologous nucleic acid sequence or/and the nucleic acid sequence coding for the desired protein are advantageously selected in order to further improve the expression level such that they have a codon usage that is at least partially adapted to the respective expression system.

Another subject matter of the invention is a reagent for producing a protein comprising
(a) a nucleic acid sequence that is heterologous to the nucleic acid sequence coding for the desired protein which can be inserted into the protein-coding nucleic acid sequence in the correct reading frame and which can form a stem-loop structure at a distance of 6-30 nucleotides on the 3′ side of the translation start codon, and
(b) an expression system that is suitable for producing the protein.

The heterologous nucleic acid sequence can be present in the form of a complete sequence or in the form of several partial sequences.

The method and reagent according to the invention can be used especially to synthesize proteins of genes that are difficult to express and to synthesize proteins starting from gene banks since the success rate can be increased compared to expression vectors that are commonly used.

The present invention is further elucidated by the following figures and examples.

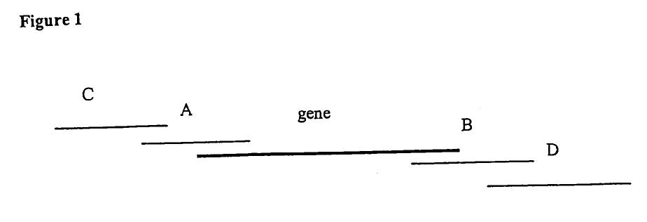

FIG. 1 shows a schematic representation of the nucleic acid sequence elements necessary for carrying out a two-step PCR.

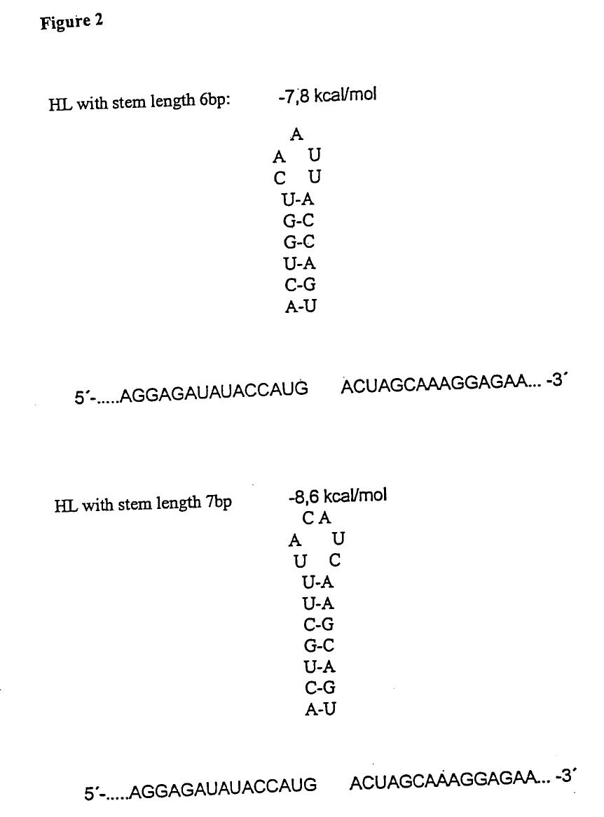

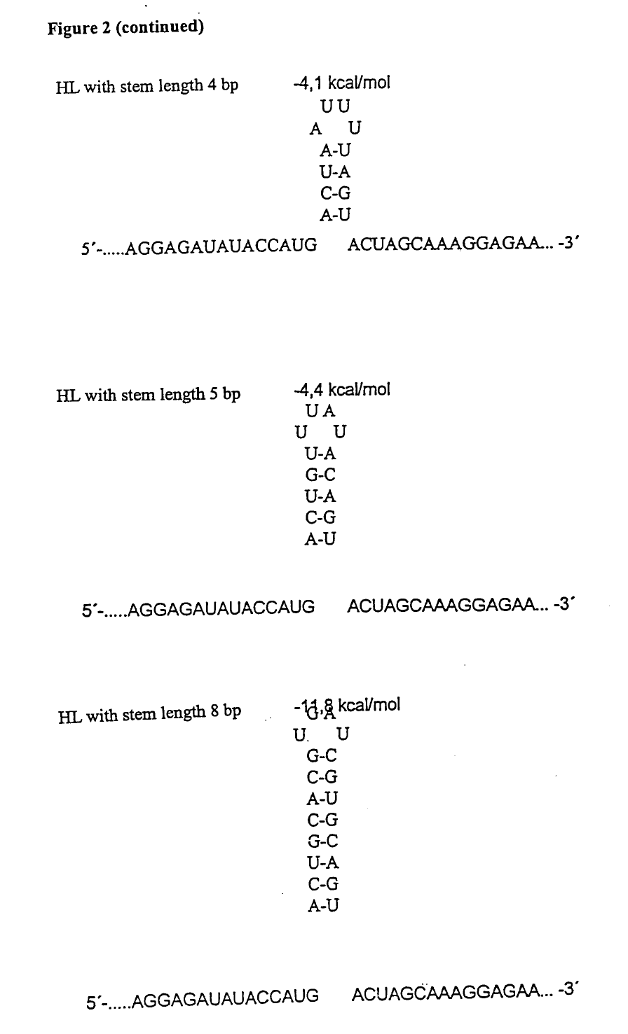

FIG. 2 shows a schematic representation of stern-loop structures of different lengths in heterologous nucleic acid sequences used for insertion into GFP expression constructs.

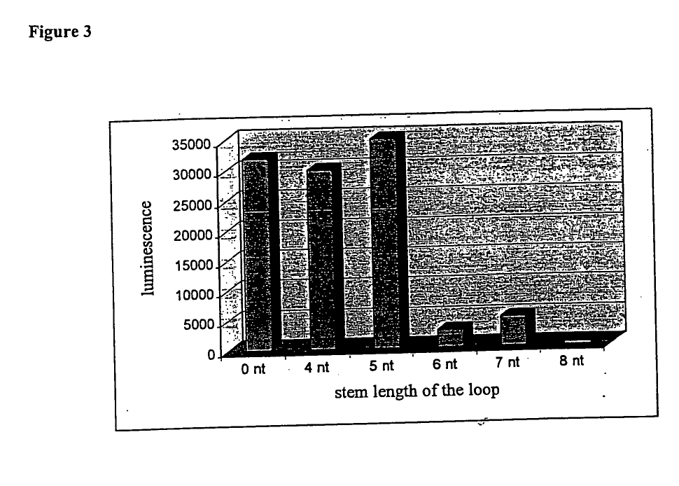

FIG. 3 shows an evaluation of the results of the expression of GFP using the hairpin-loop GFP constructs of FIG. 3 in an RTS expression system. 1 μl of each preparation (duplicate determinations) was separated electrophoretically by SDS-PAGE and blotted on a PVDF membrane. Detection was by means of a DCP Star and Lumi-Imager.

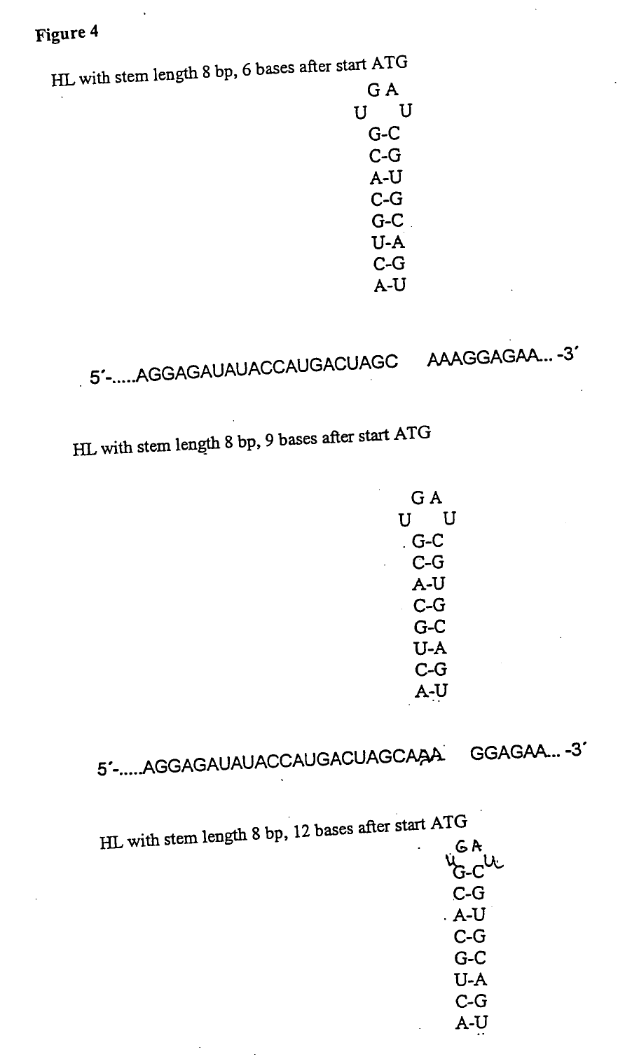

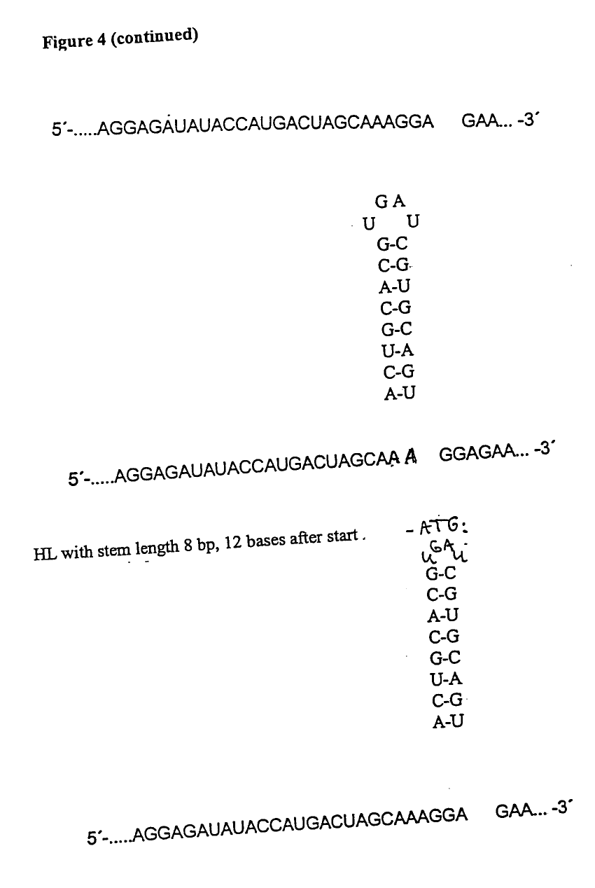

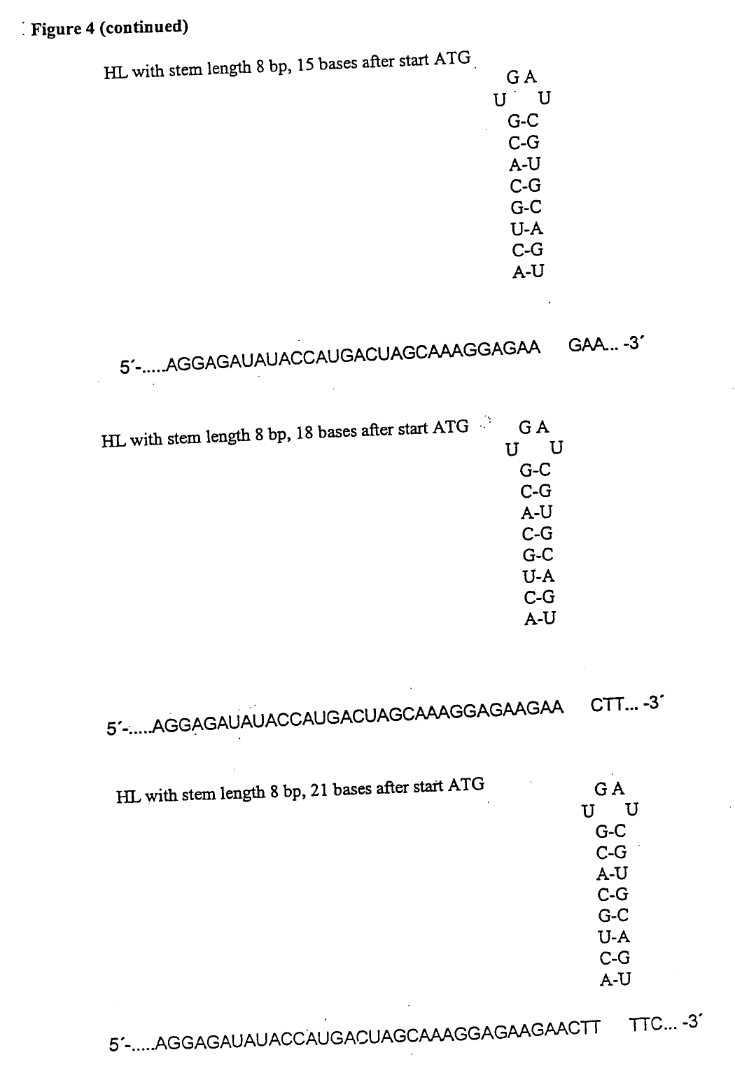

FIG. 4 shows a schematic representation of stem-loop structures at different positions in heterologous nucleic acid sequences used to insert GFP expression constructs.

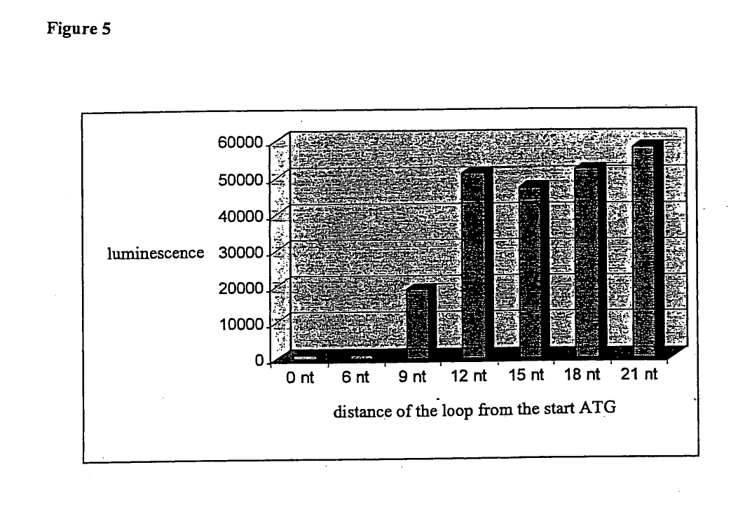

FIG. 5 shows the expression of GFP using the heterologous nucleic acid sequences shown in FIG. 4. The experiments were carried out and evaluated as described in the legend to FIG. 3.

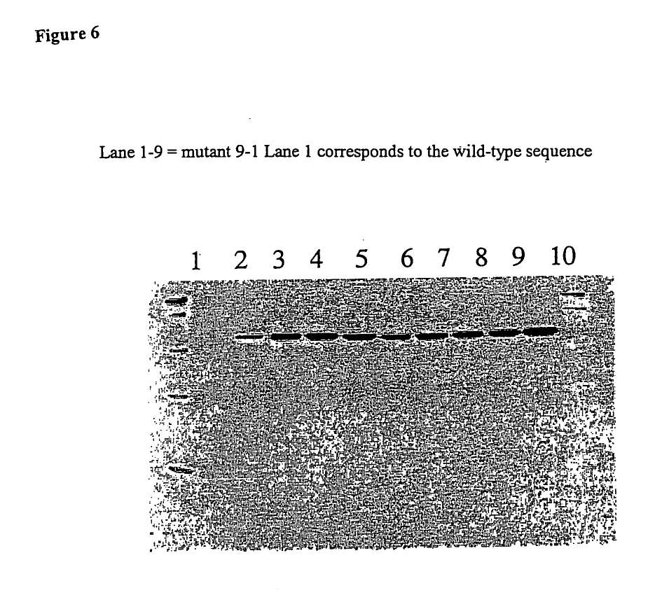

FIG. 6 shows an evaluation of the results of the expression of the CIITA gene (wild-type: lane 1; mutants lanes 2-10) using different heterologous nucleic acid sequences with stem-loop structures.

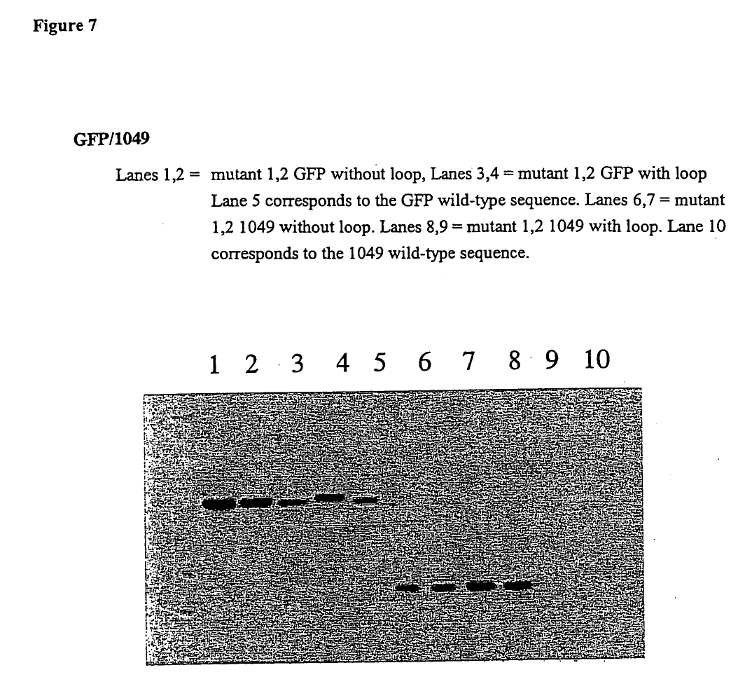

FIG. 7 shows an evaluation of the results of the expression of the CMV capsid (1049) gene (wild-type: lane 1; mutants lanes 2-10) using different heterologous nucleic acid sequences with stem-loop structures.

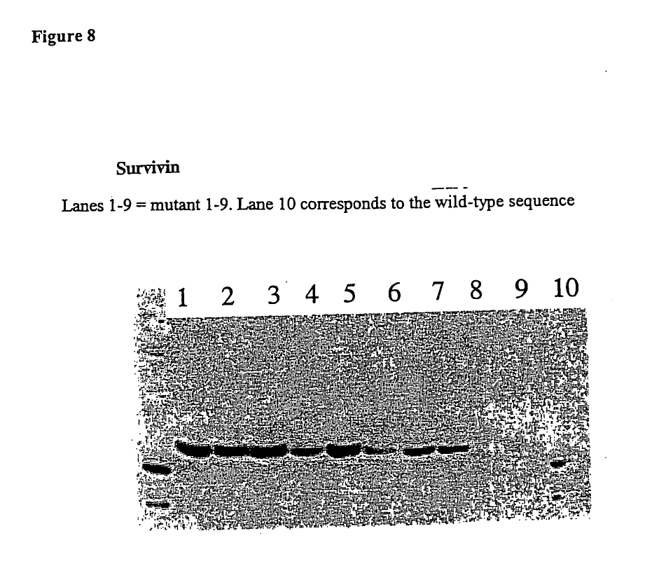

FIG. 8 shows an evaluation of the results of the expression of the survivin gene (wild-type: lane 10; mutants lanes 1-9) using different heterologous nucleic acid sequences with stem-loop structures.

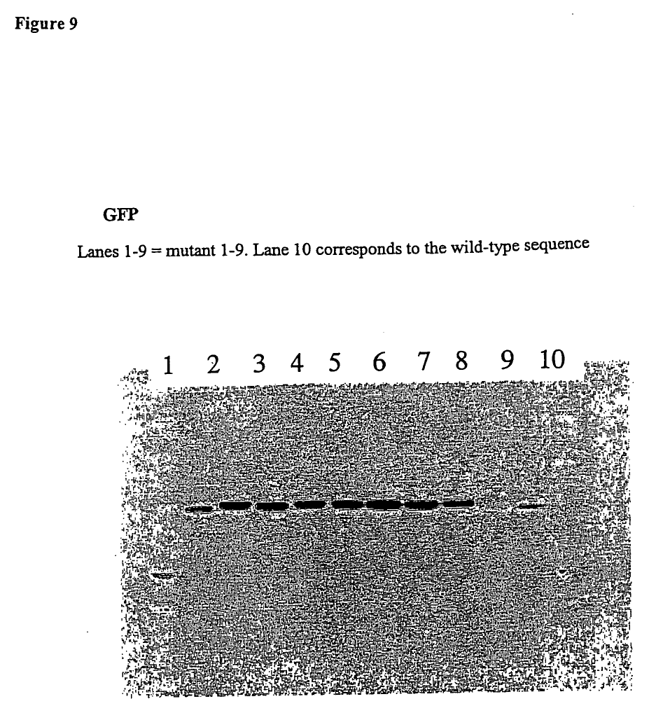

FIG. 9 shows an evaluation of the results of the expression of the GFP gene (wild-type: lane 10; mutants lanes 1-9) using different heterologous nucleic acid sequences with stem-loop structures.

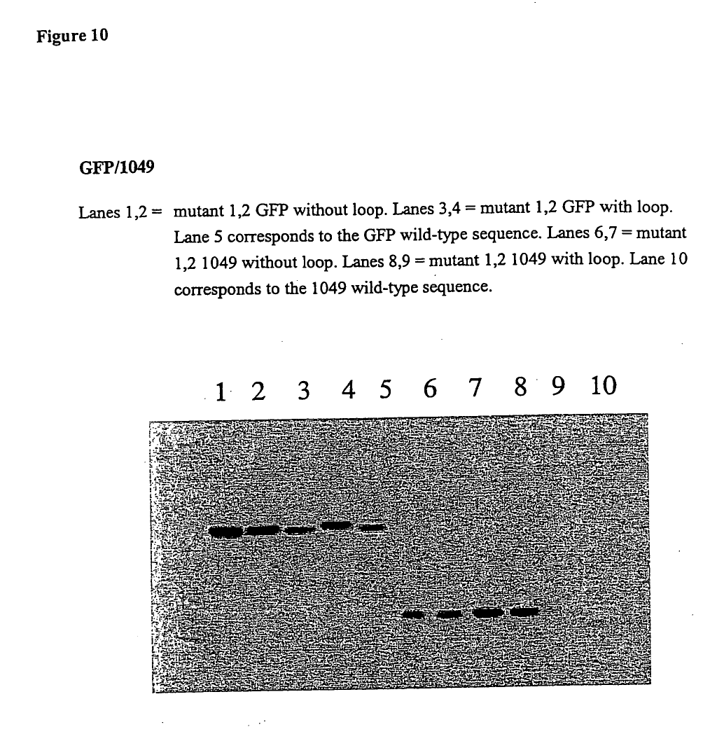

FIG. 10 shows an evaluation of the results of the expression of the GFP and the 1049 gene using different heterologous nucleic acid sequences with and without stem-loop structures.

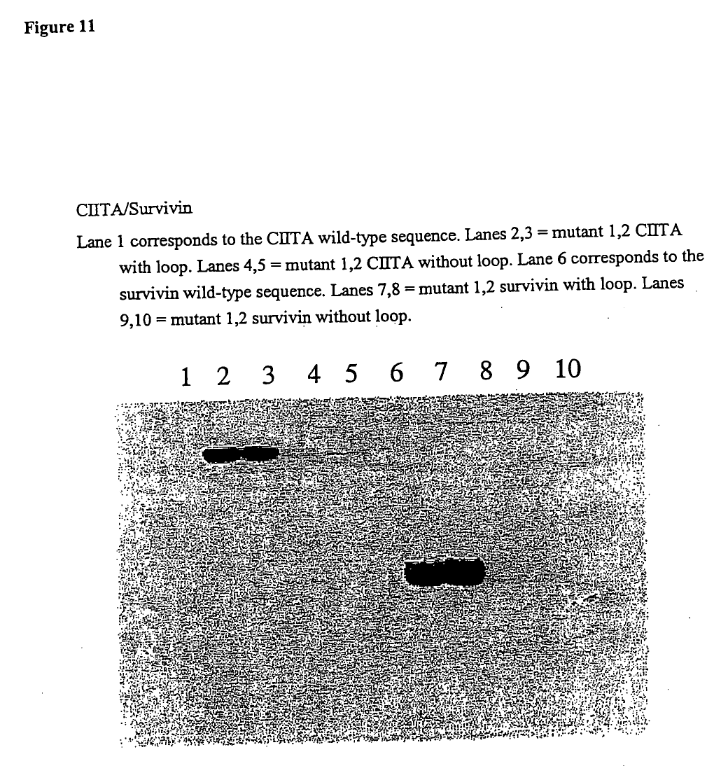

FIG. 11 shows an evaluation of the results of the expression of the CIITA and the survivin gene using different heterologous nucleic acid sequences with and without stem-loop structures.

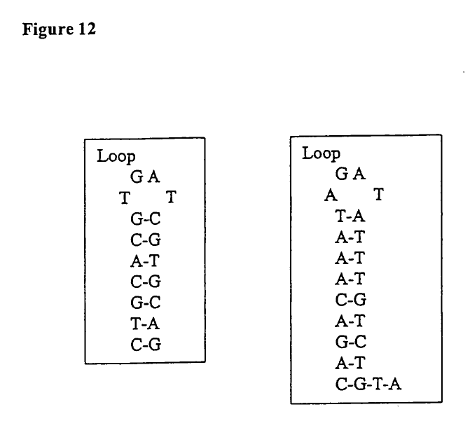

FIG. 12 shows a schematic representation of two different stem-loop structures in the heterologous sequences according to the invention.

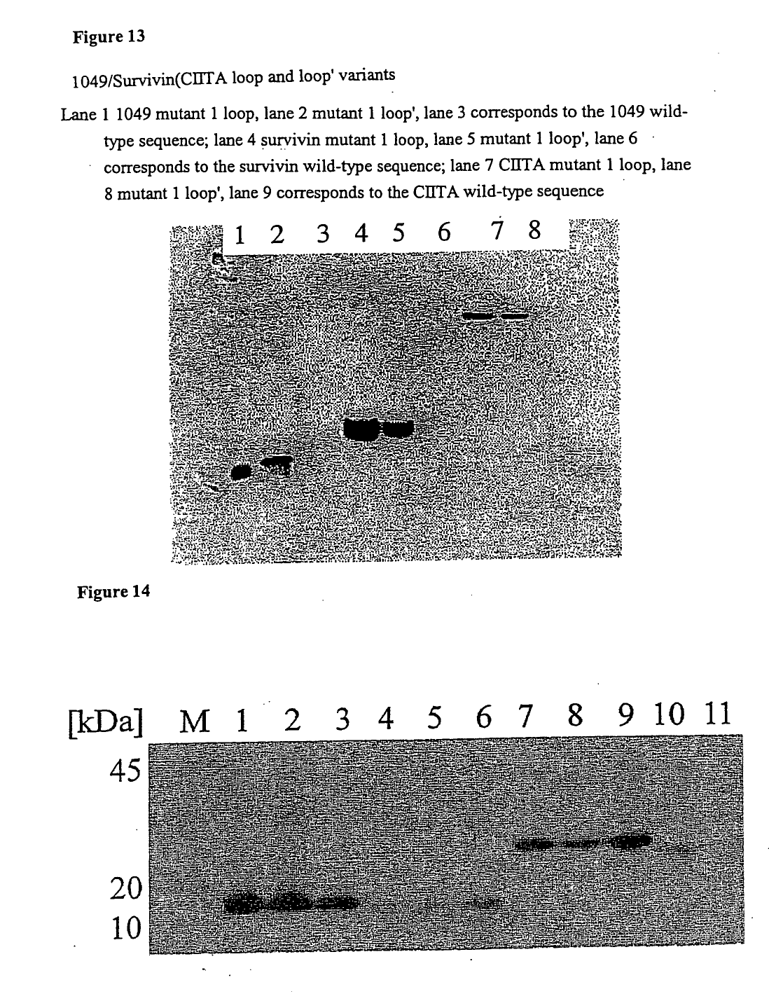

FIG. 13 shows an evaluation of the results obtained with the stem-loop structures shown in FIG. 12.

FIG. 14 shows a representation of the in vivo protein expression of RNA stem-loop constructs compared to the wild-type genes in a Western Blot. Expression of three independent clones of the RNA stem-loop mutants of the CMV capsid protein 1049 (lanes 1 to 3) and of the CMV capsid protein 1049 wild-type (lanes 4 to 6). Expression of independent clones of survivin RNA stem-loop mutants (lanes 7 to 9) and of the survivin wild-type (lanes 10, 11).

### EXAMPLES

### Example 1

### Two-Step PCR

A two-step PCR can be used to amplify genes that are to be expressed and to provide them with the appropriate control regions such as the T7 promoter, T7 gene 10 leader (g10), ribosomal binding site (RBS) and T7 terminator. In the first step the gene is amplified by means of a pair of primers (A, B) which are each complementary over a length of 15 bases with the corresponding gene and contain 15 additional bases which are complementary to a second primer pair (C, D). The second primer pair contains all important regulatory elements which are thus attached to the gene in a second PCR amplification (see FIG. 1).

The A primer can be used in this method to introduce modifications in the 5′ region of the gene. In the case of the hairpin loop constructs this A primer was used to insert hairpin loops having different lengths of the hairpin loop stem into the gene sequence at different positions behind the start codon.

Primer C

(SEQ ID NO. 1)

```
                        T7 promoter
5′-GAAATTAATACGACTCACTATAGGGAGACCACAACGGTTTCCCTCT
                                g10 RBS
AGAAATAATTTTGTTTAACTTTAAGAAGGAGATATACC-3′
complementary to A
Primer D
```

(SEQ ID NO. 2)

T7 terminator
5′-CAAAAAACCCCTCAAGACCCGTTTAGAGGCCCCAAGGGGGGCCGCC
AGTGTGCTGAATTCGCCTTTTATTA-3′
complementary to B

Reaction Conditions

The PCR reactions were usually carried out according to the following scheme using the Expand High Fidelity Kit (Roche Applied Science) on a 50 μl scale:

PCR 1:

template 10 ng/mixture; primer A 20 pmol/mixture; primer B 20 pmol/mixture
95° C. 5 min+20 times (95° C. 1 min+55 1 min+72° C. 1 min)+4° C.
   PCR 2:
2 μl PCR 1; primer C 20 pmol/mixture; primer D 20 pmol/mixture
95° C. 5 min+30 times (95° C. 1 min+50° C. 1 min+72° C. 1 min)+72° C. 10 min+4° C.

Both PCR reactions were each checked by agarose gel electrophoresis and the PCR products of the second PCR were at the same time quantified in a Lumi-Imager system with the aid of a DNA length standard which contained defined amounts of DNA. The resulting PCR products were used directly as templates in RTS expression mixtures.

### Example 2

### Expression with the RTS In Vitro Expression System

The expressions using the RTS 100 HY kit (Roche Applied Science Co.) were carried out in 50 μl batches according to the kit instructions. DNA quantities of 0.25-1 μg per reaction mixture were used. The same amounts of the respective template were always used in order to enable a comparison of the results of a series of experiments. The mixtures were incubated for 4 h at 30° C.

### Example 3

### Expression of Hairpin Loop GFP Constructs

GFP (green fluorescence protein) was used as an example to examine the effects of hairpin loops (hairpin-shaped loops) in the mRNA directly after the start ATG. For this RNA sequences were determined that form hairpin loops (HL) having different stem lengths. The longer the stem of the hairpin loop, the more energetically stable is this structure. In preparing the hairpin loops care was taken that only the codons that are found frequently in E. coli genes were used. The determined sequences for the various hairpin loops were checked by mRNA secondary structure analysis for their stability in the overall construct. Primers were prepared for sequences which had sufficient stability and these were used in the described two step PCR according to example 1.

```
Primer A:
without hairpin loop
(SEQ ID NO. 3)
        complementary to C GFP
5′-AGGAGATATACCATGACTAGCAAAGGAGAA-3′
Stem length 4 bp
(SEQ ID NO. 4)
        complementary to C HL 4 bp GFP
5′-AGGAGATATACCATGACTAATTTTAGTACTAGCAAAGGAGAA-3′
Stem length 5 bp
(SEQ ID NO. 5)
        complementary to C HL 5 bp GFP
5′-AGGAGATATACCATGACTGTTTATACAGTAACTAGCAAAGGAGAA-3′
Stem length 6 bp
(SEQ ID NO. 6)
        complementary to C HL 6 bp GFP
5′-AGGAGATATACCATGACTGGTCAATTACCAGTAACTAGCAAAGGAGAA-3′
Stem length 7 bp
(SEQ ID NO. 7)
        complementary to C HL 7 bp GFP
5′-AGGAGATATACCATGACTGCTTTACATCAAGCAGTAACTAGCAAAGGAGAA-3′
Stem length 8 bp
(SEQ ID NO. 8)
        complementary to C HL 8 bp GFP
5′-AGGAGATATACCATGACTGCACGTGATCGTGCAGTAACTAGCAAAGGAGAA-3′
Primer B
(SEQ ID NO. 9)
        complementary to D GFP
5′-ATTCGCCTTTTATTAATGATGATGATGATG-3′
```

A schematic representation of the mRNA secondary structures of the hairpin loop GFP constructs is shown in FIG. 2.

RTS Expression

After expression in the RTS according to example 2 the amount of GFP formed was measured in a fluorimeter for the purposes of verification and the Western Blot was quantitatively analysed by CDP-Star detection and evaluation in a Lumi-Imager. The results are shown in FIG. 3.

It can be clearly seen that the expression rate varies with the stem length of the hairpin loop. The expression rate is relatively constant up to a stem length of 5 bp and then subsequently decreases. Almost no expression can be detected at a stem length of 8 bp. These investigations confirm the results obtained above. Hence one can say that a hairpin loop with a stem length of more than 6 bp or rather with a free energy of −7.8 kcal/mol represents a structure which has a considerable effect on expression. This can be explained by the fact that this structure is stable under the expression conditions and thus the start ATG in front of it is not freely accessible.

### Example 4

### Determination of the Minimum Distance from the Start ATG

In order to now determine up to which distance such a hairpin loop exerts an effect on expression, the hairpin loop with the stem length of 8 bp (energy −11.8 kcal/mol) was shifted in steps of 3 bases from the start ATG into the GFP sequence. The sequences of the A primers obtained in this manner were as follows:

Stem length 8 bp, shifted 6 bases into the GFP sequence (SEQ ID NO. 10):

HL 8 bp GFP
5′-AGG...ATGACTAGCACT...GTAAAAGGAGAAGAACTT-3′

Stem length 8 bp, shifted 9 bases into the GFP sequence (SEQ ID NO. 11):

HL 8 bp GFP
5′-AGG...ATGACTAGCAAAACT...GTAGGAGAAGAACTTTTC-3′

Stem length 8 bp, shifted 12 bases into the GFP sequence (SEQ ID NO. 12):

HL 8 bp GFP
5′-AGG...ATGACTAGCAAAGGAACT...GTAGAAGAACTTTT
CACT-3′

Stem length 8 bp, shifted 15 bases into the GFP sequence (SEQ ID NO. 13):

HL 8 bp GFP
5′-AGG...ATGACTAGCAAAGGAGAAACT...GTAGAACTTTTCACTGG
A-3′

Stem length 8 bp, shifted 18 bases into the GFP sequence (SEQ ID NO. 14):

HL 8 bp GFP
5′-AGG...ATGACTAGCAAAGGAGAAGAAACT...GTACTTTTCACTGG
AGTT-3′

Stem length 8 bp, shifted 21 based into the GFP sequence (SEQ ID NO. 15):

HL 8 bp GFP
5′-AGG...ATGACTAGCAAAGGAGAAGAACTTACT...GTATTCACTGG
AGTTGTC-3′

These DNA constructs with the secondary structures shown in FIG. 4 were also synthesized by a two-step PCR using the previously described primers B, C and D and used directly from the PCR reaction as templates in expression preparations. It was ensured that the same amounts of template were used by quantification on an agarose gel with the DNA marker VII and evaluation of this gel in a Lumi-Imager. The expression mixtures were evaluated by a Western Blot. The results are shown in FIG. 5.

The expressions show that mRNA translation is possible at a distance of more than 9 bases from the start ATG. There is still an inhibitory effect of the hairpin loop. The translation does not proceed almost uninhibited until the distance exceeds 12 bases. Hence one can conclude from these results that the ribosome requires a space of 9-11 bases after the start ATG. Furthermore, it may be deduced from these results that a hairpin loop which is 12 or more bases distant from the start ATG has an effect on the mRNA secondary structure but no effect on the initiation of expression.

### Example 5

### Introduction of Stem-Loop Structures to Break Down Unfavourable Secondary Structures

In earlier expression experiments using the Rapid Translation System (Roche Applied Science) only a low or even no expression was found for some genes. The cause was often determined to be an unfavourable RNA secondary structure in which either the start codon or the Shine-Dalgarno sequence was involved in a secondary structure with the gene sequence and was thus present in a bound form.

A heterologous nucleic acid sequence with a hairpin loop and a stem length of 7 bases at a distance of 15 bases after the start codon was introduced for three of these genes, survivin, cytomegalovirus capsid protein 1049 (1049) and Class II transactivator (CIITA). The wild-type gene (see below *) without the start ATG was placed directly after the hairpin loop. AT-rich sequences were placed in front of the hairpin loop which are able to form less stable base pairs than GC-rich sequences. Furthermore, care was taken that no rare codons for E. coli were used within the introduced sequences.

Due to the fact that, on the one hand, a stable ideal hairpin loop is already present and, on the other hand, a sequence follows directly after the start codon which has no tendency to form secondary structures, the initiation complex with the small ribosomal subunit should have free access to the Shine-Dalgarno sequence and the start ATG independently of the subsequent gene.

9 different AT-rich sequences were used in front of the hairpin loops and compared with the wild-type genes *. The GFP cycle 3 protein with the same hairpin loops and AT-rich sequences was synthesized as a control gene by the two-step PCR mentioned in example 1. The sequences of the A and B primers are shown below. The homologous regions to primer C are underlined in primer 1. The AT-rich sequence is shown in italics, the hairpin loop is shown in bold type and the wild-type gene sequence is shown in bold type and underlined. In primer B the regions that are homologous to primer D are underlined and the regions that have a homology to the wild-type gene are shown in bold type. In contrast to example 1 the following primer was used as primer D:

Primer D (SEQ ID NO. 16):
CAAAAAACCCCTCAAGACCCGTTTAGAGGCCCCAAGGGGTTGGGAGTAGA
ATGTTAAGGATTAGTTTATTA

The underlined region is homologous to primer C.

Variants of primer A:

1049-1 (SEQ ID NO. 17):
AGGAGATATACCATGAAATATACATATTCTCTGCACGTGATCGTGCAG
GCTAACACCGCG
1049-2 (SEQ ID NO: 18):
AGGAGATATACCATGAAAACATATTATTCTCTGCACGTGATCGTGCAGG
CTAACACCGCG
1049-3 (SEQ ID NO: 19):
AGGAGATATACCATGAAATATTCTTATACACTGCACGTGATCGTGCAGG
CTAACACCGCG
1049-4 (SEQ ID NO: 20):
AGGAGATATACCATGAAATATTATTCTACACTGCACGTGATCGTGCAGG
CTAACACCGCG
1049-5 (SEQ ID NO: 21):
AGGAGATATACCATGAAATATACATATTCACTGCACGTGATCGTGCAGG
CTAACACCGCG
1049-6 (SEQ ID NO: 22):
AGGAGATATACCATGAAAACATATTATTCACTGCACGTGATCGTGCAGG
CTAACACCGCG
1049-7 (SEQ ID NO: 23):
AGGAGATATACCATGAAATATTCATATACACTGCACGTGATCGTGCAGG
CTAACACCGCG
1049-8 (SEQ ID NO: 24):
AGGAGATATACCATGAAATATTATTCAACACTGCACGTGATCGTGCAGG
CTAACACCGCG
1049-9 (SEQ ID NO: 25):
AGGAGATATACCATGCATCATCATCATCATCTGCACGTGATCGTGCAGG
CTAACACCGCG
1049-10 (wild-type) (SEQ ID NO: 26):
AGGAGATATACCATGGCTAACACCGCG
1049-primer B (SEQ ID NO: 27):
AGGATTAGTTTATTAATGATGATGATGATGATGGCGCCGGGTGCGCGA
The underlined is homologous to primer D
Variants of primer A:
Survivin-1 (SEQ ID NO. 28):
AGGAGATATACCATGAAATATACATATTCTCTGCACGTGATCGTGCAG
GGTGCCCCGACG
Survivin-2 (SEQ ID NO. 29):
AGGAGATATACCATGAAAACATATTATTCTCTGCACGTGATCGTGCAGG
GTGCCCCGACG
Survivin-3 (SEQ ID NO. 30):
AGGAGATATACCATGAAATATTCTTATACACTGCACGTGATCGTGCAGG
GTGCCCCGACG
Survivin-4 (SEQ ID NO. 31):
AGGAGATATACCATGAAATATTATTCTACACTGCACGTGATCGTGCAGG
GTGCCCCGACG
Survivin-5 (SEQ ID NO. 32):
AGGAGATATACCATGAAATATACATATTCACTGCACGTGATCGTGCAGG
GTGCCCCGACG
Survivin-6 (SEQ ID NO. 33):
AGGAGATATACCATGAAAACATATTATTCACTGCACGTGATCGTGCAGG
GTGCCCCGACG
Survivin-7 (SEQ ID NO. 34):
AGGAGATATACCATGAAATATTCATATACACTGCACGTGATCGTGCAGG
GTGCCCCGACG
Survivin-8 (SEQ ID NO. 35):
AGGAGATATACCATGAAATATTATTCAACACTGCACGTGATCGTGCAGG
GTGCCCCGACG
Survivin-9 (SEQ ID NO. 36):
AGGAGATATACCATGCATCATCATCATCATCTGCACGTGATCGTGCAGG
GTGCCCCGACG
Survivin-10 (A wild-type) (SEQ ID NO. 37):
AGGAGATATACCATGGGTGCCCCGACG
Survivin-primer B (SEQ ID NO. 38):
AGGATTAGTTTATTAATGATGATGATGATGATGATCCATGGCAGCCAGC
CIITA-1 (SEQ ID NO. 39):
AGGAGATATACCATGAAATATACATATTCTCTGCACGTGATCGTGCAG
GAGTTGGGGCCC
CIITA-2 (SEQ ID NO. 40):
AGGAGATATACCATGAAAACATATTATTCTCTGCACGTGATCGTGCAGG
AGTTGGGGCCC
CIITA -3 (SEQ ID NO. 41):
AGGAGATATACCATGAAATATTCTTATACACTGCACGTGATCGTGCAGG
AGTTGGGGCCC
CIITA-4 (SEQ ID NO. 42):
AGGAGATATACCATGAAATATTATTCTACACTGCACGTGATCGTGCAGG
AGTTGGGGCCC
CIITA-5 (SEQ ID NO. 43):
AGGAGATATACCATGAAATATACATATTCACTGCACGTGATCGTGCAGG
AGTTGGGGCCC
CIITA-6 (SEQ ID NO. 44):
AGGAGATATACCATGAAAACATATTATTCACTGCACGTGATCGTGCAGG
AGTTGGGGCCC
CIITA-7 (SEQ lID NO. 45):
AGGAGATATACCATGAAATATTCATATACACTGCACGTGATCGTGCAGG
AGTTGGGGCCC
CIITA-8 (SEQ ID NO. 46):
AGGAGATATACCATGAAATATTATTCAACACTGCACGTGATCGTGCAGG
AGTTGGGGCCC
CIITA-9 (SEQ ID NO. 47):
AGGAGATATACCATGCATCATCATCATCATCTGCACGTGATCGTGCAGG
AGTTGGGGCCC
CIITA-10 (A wild-type) (SEQ ID NO. 48):
AGGAGATATACCATGGAGTTGGGGCCC
CIITA-primer B (SEQ ID NO. 49):
AGGATTAGTTTATTATTAATGATGATGATGATGATGAGAACCCCC

The sequences of the expression constructs for mutant 1 and the wild-type generated by PCR are shown in the following. The wild-type gene sequence is shown in bold type. A hexa-histidine tag was inserted at the end of the gene using the B primer to enable detection with a specific antibody (underlined).

1049 - 1 (431 bp) (SEQ ID NO. 50):
GAAATTAATACGACTCACTATAGGGAGACCACAACGGTTTCCCTCTAGAA
ATAATTTTGTTTAACTTTAAGAAGGAGATATACCATGAAATATACATATT
CTCTGCACGTGATCGTGCAGGCTAACACCGCGCCGGGACCCACGGTGGCC
AACAAGCGGGACGAAAAACACCGTCACGTCGTTAACGTCGTTTTGGAGCT
GCCGACCGAGATATCAGAGGCCACCCACCCGGTGTTGGCCACCATGCTGA
GCAAGTACACGCGCATGTCCAGCCTGTTTAATGACAAGTGCGCCTTTAAG
CTGGACCTGTTGCGCATGGTAGCCGTGTCGCGCACCCGGCGCCATCATCA
TCATCATCATTAATAAACTAATCCTTAACATTCTACTCCCAACCCCTTGG
GGCCTCTAAACGGGTCTTGAGGGGTTTTTTG
1049 - 10 (wild-type) (398 bp) SEQ ID NO. 51):
GAAATTAATACGACTCACTATAGGGAGACCACAACGGTTTCCCTCTAGAA
ATAATTTTGTTTAACTTTAAGAAGGAGATATACCATGGCTAACACCGCGC
CGGGACCCACGGTGGCCAACAAGCGGGACGAAAAACACCGTCACGTCGTT
AACGTCGTTTTGGAGCTGCCGACCGAGATATCAGAGGCCACCCACCCGGT
GTTGGCCACCATGCTGAGCAAGTACACGCGCATGTCCAGCCTGTTTAATG
ACAAGTGCGCCTTTAAGCTGGACCTGTTGCGCATGGTAGCCGTGTCGCGC
ACCCGGCGCCATCATCATCATCATCATTAATAAACTAATCCTTAACATTC
TACTCCCAACCCCTTGGGGCCTCTAAACGGGTCTTGAGGGGTTTTTTG
Survivin - 1 (632 bp) (SEQ ID NO. 52):
GAAATTAATACGACTCACTATAGGGAGACCACAACGGTTTCCCTCTAGAA
ATAATTTTGTTTAACTTTAAGAAGGAGATATACCATGAAATATACATATT
CTCTGCACGTGATCGTGCAGGGTGCCCCGACGTTGCCCCCTGCCTGGCAG
CCCTTTCTCAAGGACCACCGCATCTCTACATTCAAGAACTGGCCCTTCTT
GGAGGGCTGCGCCTGCACCCCGGAGCGGATGGCCGAGGCTGGCTTCATCC
ACTGCCCCACTGAGAACGAGCCAGACTTGGCCCAGTGTTTCTTCTGCTTC
AAGGAGCTGGAAGGCTGGGAGCCAGATGACGACCCCATAGAGGAACATAA
AAAGCATTCGTCCGGTTGCGCTTTCCTTTCTGTCAAGAAGCAGTTTGAAG
AATTAACCCTTGGTGAATTTTTGAAACTGGACAGAGAAAGAGCCAAGAAC
AAAATTGCAAAGGAAACCAACAATAAGAAGAAAGAATTTGAGGAAACTGC
GAAGAAAGTGCGCCGTGCCATCGAGCAGCTGGCTGCCATGGATCATCATC
ATCATCATCATTAATAAACTAATCCTTAACATTCTACTCCCAACCCCTTG
GGGCCTCTAAACGGGTCTTGAGGGGTTTTTTG
Survivin - 10 (wild type) (599 bp) (SEQ ID NO. 53):
GAAATTAATACGACTCACTATAGGGAGACCACAACGGTTTCCCTCTAGAA
ATAATTTTGTTTAACTTTAAGAAGGAGATATACCATGGGTGCCCCGACGT
TGCCCCCTGCCTGGCAGCCCTTTCTCAAGGACCACCGCATCTCTACATTC
AAGAACTGGCCCTTCTTGGAGGGCTGCGCCTGCACCCCGGAGCGGATGGC
CGAGGCTGGCTTCATCCACTGCCCCACTGAGAACGAGCCAGACTTGGCCC
AGTGTTTCTTCTGCTTCAAGGAGCTGGAAGGCTGGGAGCCAGATGACGAC
CCCATAGAGGAACATAAAAAGCATTCGTCCGGTTGCGCTTTCCTTTCTGT
CAAGAAGCAGTTTGAAGAATTAACCCTTGGTGAATTTTTGAAACTGGACA
GAGAAAGAGCCAAGAACAAAATTGCAAAGGAAACCAACAATAAGAAGAAA
GAATTTGAGGAAACTGCGAAGAAAGTGCGCCGTGCCATCGAGCAGCTGGC
TGCCATGGATCATCATCATCATCATCATTAATAAACTAATCCTTAACATT
CTACTCCCAACCCCTTGGGGCCTCTAAACGGGTCTTGAGGGGTTTTTTG
CIITA - 1 (1400 bp) (SEQ ID NO. 54):
GAAATTAATACGACTCACTATAGGGAGACCACAACGGTTTCCCTCTAGAA
ATAATTTTGTTTAACTTTAAGAAGGAGATATACCATGAAATATACATATT
CTCTGCACGTGATCGTGCAGGAGTTGGGGCCCCTAGAAGGTGGCTACCTG
GAGCTTCTTAACAGCGATGCTGACCCCCTGTGCCTCTACCACTTCTATGA
CCAGATGGACCTGGCTGGAGAAGAAGAGATTGAGCTCTACTCAGAACCCG
ACACAGACACCATCAACTGCGACCAGTTCAGCAGGCTGTTGTGTGACATG
GAAGGTGATGAAGAGACCAGGGAGGCTTATGCCAATATCGCGGAACTGGA
CCAGTATGTCTTCCAGGACTCCCAGCTGGAGGGCCTGAGCAAGGACATTT
TCAAGCACATAGGACCAGATGAAGTGATCGGTGAGAGTATGGAGATGCCA
GCAGAAGTTGGGCAGAAAAGTCAGAAAAGACCCTTCCCAGAGGAGCTTCC
GGCAGACCTGAAGCACTGGAAGCCAGCTGAGCCCCCCACTGTGGTGACTG
GCAGTCTCCTAGTGGGACCAGTGAGCGACTGCTCCACCCTGCCCTGCCTG
CCACTGCCTGCGCTGTTCAACCAGGAGCCAGCCTCCGGCCAGATGCGCCT
GGAGAAAACCGACCAGATTCCCATGCCTTTCTCCAGTTCCTCGTTGAGCT
GCCTGAATCTCCCTGAGGGACCCATCCAGTTTGTCCCCACCATCTCCACT
CTGCCCCATGGGCTCTGGCAAATCTCTGAGGCTGGAACAGGGGTCTCCAG
TATATTCATCTACCATGGTGAGGTGCCCCAGGCCAGCCAAGTACCCCCTC
CCAGTGGATTCACTGTCCACGGCCTCCCAACATCTCCAGACCGGCCAGGC
TCCACCAGCCCCTTCGCTCCATCAGCCACTGACCTGCCCAGCATGCCTGA
ACCTGCCCTGACCTCCCGAGCAAACATGACAGAGCACAAGACGTCCCCCA
CCCAATGCCCGGCAGCTGGAGAGGTCTCCAACAAGCTTCCAAAATGGCCT
GAGCCGGTGGAGCAGTTCTACCGCTCACTGCAGGACACGTATGGTGCCGA
GCCCGCAGGCCCGGATGGCATCCTAGTGGAGGTGGATCTGGTGCAGGCCA
GGCTGGAGAGGAGCAGCAGCAAGAGCCTGGAGCGGGAACTGGCCACCCCG
GACTGGGCAGAACGGCAGCTGGCCCAAGGAGGCCTGGCTGAGGTGCTGTT
GGCTGCCAAGGAGCACCGGCGGCCGCGTCGACTCGAGCGAGCTCCCGGGG
GGGGTTCTCATCATCATCATCATCATTAATAATAAACTAATCCTTAACAT
TCTACTCCCAACCCCTTGGGGCCTCTAAACGGGTCTTGAGGGGTTTTTTG
CIITA - 10 WT 1367 bp (SEQ ID NO. 55):
GAAATTAATACGACTCACTATAGGGAGACCACAACGGTTTCCCTCTAGAA
ATAATTTTGTTTAACTTTAAGAAGGAGATATACCATGGAGTTGGGGCCCC
TAGAAGGTGGCTACCTGGAGCTTCTTAACAGCGATGCTGACCCCCTGTGC
CTCTACCACTTCTATGACCAGATGGACCTGGCTGGAGAAGAAGAGATTGA
GCTCTACTCAGAACCCGACACAGACACCATCAACTGCGACCAGTTCAGCA
GGCTGTTGTGTGACATGGAAGGTGATGAAGAGACCAGGGAGGCTTATGCC
AATATCGCGGAACTGGACCAGTATGTCTTCCAGGACTCCCAGCTGGAGGG
CCTGAGCAAGGACATTTTCAAGCACATAGGACCAGATGAAGTGATCGGTG
AGAGTATGGAGATGCCAGCAGAAGTTGGGCAGAAAAGTCAGAAAAGACCC
TTCCCAGAGGAGCTTCCGGCAGACCTGAAGCACTGGAAGCCAGCTGAGCC
CCCCACTGTGGTGACTGGCAGTCTCCTAGTGGGACCAGTGAGCGACTGCT
CCACCCTGCCCTGCCTGCCACTGCCTGCGCTGTTCAACCAGGAGCCAGCC
TCCGGCCAGATGCGCCTGGAGAAAACCGACCAGATTCCCATGCCTTTCTC
CAGTTCCTCGTTGAGCTGCCTGAATCTCCCTGAGGGACCCATCCAGTTTG
TCCCCACCATCTCCACTCTGCCCCATGGGCTCTGGCAAATCTCTGAGGCT
GGAACAGGGGTCTCCAGTATATTCATCTACCATGGTGAGGTGCCCCAGGC
CAGCCAAGTACCCCCTCCCAGTGGATTCACTGTCCACGGCCTCCCAACAT
CTCCAGACCGGCCAGGCTCCACCAGCCCCTTCGCTCCATCAGCCACTGAC
CTGCCCAGCATGCCTGAACCTGCCCTGACCTCCCGAGCAAACATGACAGA
GCACAAGACGTCCCCCACCCAATGCCCGGCAGCTGGAGAGGTCTCCAACA
AGCTTCCAAAATGGCCTGAGCCGGTGGAGCAGTTCTACCGCTCACTGCAG
GACACGTATGGTGCCGAGCCCGCAGGCCCGGATGGCATCCTAGTGGAGGT
GGATCTGGTGCAGGCCAGGCTGGAGAGGAGCAGCAGCAAGAGCCTGGAGC
GGGAACTGGCCACCCCGGACTGGGCAGAACGGCAGCTGGCCCAAGGAGGC
CTGGCTGAGGTGCTGTTGGCTGCCAAGGAGCACCGGCGGCCGCGTCGACT
CGAGCGAGCTCCCGGGGGGGGTTCTCATCATCATCATCATCATTAATAAT
AAACTAATCCTTAACATTCTACTCCCAACCCCTTGGGGCCTCTAAACGGG
TCTTGAGGGGTTTTTTG
GFP CyC3 - 1 (938 bp) (SEQ ID NO. 56):
GAAATTAATACGACTCACTATAGGGAGACCACAACGGTTTCCCTCTAGAA
ATAATTTTGTTTAACTTTAAGAAGGAGATATACCATGAAATATACATATT
CTCTGCACGTGATCGTGCAGACTAGCAAAGGAGAAGAACTTTTCACTGGA
GTTGTCCCAATTCTTGTTGAATTAGATGGTGATGTTAATGGGCACAAATT
TTCTGTCAGTGGAGAGGGTGAAGGTGATGCTACATACGGAAAGCTTACCC
TTAAATTTATTTGCACTACTGGAAAACTACCTGTTCCATGGCCAACACTT
GTCACTACTTTCTCTTATGGTGTTCAATGCTTTTCCCGTTATCCGGATCA
TATGAAACGGCATGACTTTTTCAAGAGTGCCATGCCCGAAGGTTATGTAC
AGGAACGCACTATATCTTTCAAAGATGACGGGAACTACAAGACGCGTGCT
GAAGTCAAGTTTGAAGGTGATACCCTTGTTAATCGTATCGAGTTAAAAGG
TATTGATTTTAAAGAAGATGGAAACATTCTCGGACACAAACTCGAGTACA
ACTATAACTCACACAATGTATACATCACGGCAGACAAACAAAAGAATGGA
ATCAAAGCTAACTTCAAAATTCGCCACAACATTGAAGATGGATCCGTTCA
ACTAGCAGACCATTATCAACAAAATACTCCAATTGGCGATGGCCCTGTCC
TTTTACCAGACAACCATTACCTGTCGACACAATCTGCCCTTTCGAAAGAT
CCCAACGAAAAGAGAGACCACATGGTCCTTCTTGAGTTTGTAACAGCTGC
TGGGATTACACATGGCATGGATGAACTATACAAACCCGGGGGGGGTTCTC
ATCATCATCATCATCATTAATAAACTAATCCTTAACATTCTACTCCCAAC
CCCTTGGGGCCTCTAAACGGGTCTTGAGGGGTTTTTTG
pIVEX-GFP CyC3 - 10 (905 bp) (SEQ ID NO. 57):
GAAATTAATACGACTCACTATAGGGAGACCACAACGGTTTCCCTCTAGAA
ATAATTTTGTTTAACTTTAAGAAGGAGATATACCATGACTAGCAAAGGAG
AAGAACTTTTCACTGGAGTTGTCCCAATTCTTGTTGAATTAGATGGTGAT
GTTAATGGGCACAAATTTTCTGTCAGTGGAGAGGGTGAAGGTGATGCTAC
ATACGGAAAGCTTACCCTTAAATTTATTTGCACTACTGGAAAACTACCTG
TTCCATGGCCAACACTTGTCACTACTTTCTCTTATGGTGTTCAATGCTTT
TCCCGTTATCCGGATCATATGAAACGGCATGACTTTTTCAAGAGTGCCAT
GCCCGAAGGTTATGTACAGGAACGCACTATATCTTTCAAAGATGACGGGA
ACTACAAGACGCGTGCTGAAGTCAAGTTTGAAGGTGATACCCTTGTTAAT
CGTATCGAGTTAAAAGGTATTGATTTTAAAGAAGATGGAAACATTCTCGG
ACACAAACTCGAGTACAACTATAACTCACACAATGTATACATCACGGCAG
ACAAACAAAAGAATGGAATCAAAGCTAACTTCAAAATTCGCCACAACATT
GAAGATGGATCCGTTCAACTAGCAGACCATTATCAACAAAATACTCCAAT
TGGCGATGGCCCTGTCCTTTTACCAGACAACCATTACCTGTCGACACAAT
CTGCCCTTTCGAAAGATCCCAACGAAAAGAGAGACCACATGGTCCTTCTT
GAGTTTGTAACAGCTGCTGGGATTACACATGGCATGGATGAACTATACAA
ACCCGGGGGGGGTTCTCATCATCATCATCATCATTAATAAACTAATCCTT
AACATTCTACTCCCAACCCCTTGGGGCCTCTAAACGGGTCTTGAGGGGTT
TTTTG

The expressions shown in FIGS. 6 to 9 show that DNA templates synthesized with the stem-loop structures in all cases resulted in protein synthesis whereas no protein synthesis took place with the wild-type gene. The expression of mutant 9 with the hexa-histidine sequence is not quite as good as that of the other AT-rich sequences but has the advantage that the protein that is formed can be purified on Ni-NTA chelate columns by means of this six histidine residue label. Even in the case of the GFP gene which is a gene that is in any case expressed well, the stem-loop constructs resulted in an increase in yield.

### Example 6

### Removal of the Stem-Loop Structure to Prove its Function

In order to differentiate between the effect of the stem-loop structure and the effect of the introduced AT-rich sequence, an identical PCR was produced of each of the two mutants but without the stem-loop part and expressed in a direct comparison with the stem-loop mutants.

These examples clearly show the effect of the stem-loop structure. Whereas in the case of GFP the AT-rich sequence alone increases expression, the stem-loop sequence makes the decisive contribution in the case of genes that are difficult to express.

### Example 7

### Modification of the Stem-Loop Structure to Determine the Important Properties for its Function

In order to determine the effect of GC bases within the stem-loop structure, their sequence was replaced by an AT-rich sequence having the same free energy as that of the GC-rich stem-loop.

For this a new stem-loop (loop′) having the sequence CAG.ACA.AAT.AGA.TAT. TTG.TCT.GTA (G=−9.8 kcal/mol and a stem length of 9 base pairs) was combined with the AT-rich sequence of mutant 1 instead of the original stem-loop sequence CTG.CAC.GTG.ATC.GTG.CAG (G=−9.8 kcal/mol and a stem length of 7 base pairs) for the examples survivin, CIITA and 1049. The two structures are shown in FIG. 12.

It can be seen that the two stem-loop variants considerably increase expression compared to the respective wild-type genes or enable expression for the first time. The GC-rich stem-loop variants exhibit a slightly more pronounced increase in expression.

### Example 8

### In Vivo Protein Expression

PCR products from example 5 with the expression construct for the wild-type gene of the cytomegalovirus capsid protein 1049 as well as for the survivin wild-type gene were cloned into pBAD-TOPO (Invitrogen, Carlsbad, USA) vectors.

Expression constructs for mutants of the gene for the cytomegalovirus capsid protein 1049 and for the survivin gene were also cloned into this vector. Afterwards the plasmids were transformed into B21 pLyS strains (Stratagene, Amsterdam, Netherlands) and steaked onto LB plates containing 100 μg/ml carbenicillin and 34 μg/ml chloroamphenicol. The inserts were checked by sequencing. Three colonies of each were isolated for the in vivo protein expression and grown for 5 h in 4 ml medium at 37° C. When the cell density reached 10⁸ cells/ml, the expression was induced by adding 1 mM IPTG and they were incubated for a further 2 hours. 1 ml of each cell suspension was centrifuged (3 min at 14000 rpm) and the precipitate was heated on a thermoshaker in SDS sample buffer for 20 min at 95° C. and 1400 rpm. 10 μl aliquots were applied to an SDS gel and analysed with a Western Blot as described in example 5.

The expressions in FIG. 14 show that the stem-loop constructs for the two examined gene cytomegalovirus capsid protein 1049 as well as survivin also exhibited a substantially higher expression in vivo than the wild-type genes. This proves that the results of the in vitro expression can also be applied to in vivo expression.

1. A method for producing a protein, the method comprising the steps of:
(a) providing a nucleic acid sequence coding for the protein wherein the nucleic acid sequence coding for the protein comprises a translation start codon;
(b) inserting a heterologous nucleic acid sequence on the 3′ side of the translation start codon in the correct reading frame, wherein said heterologous nucleic acid sequence forms a stem-loop structure on the 3′ side of the translation start codon 6-30 nucleotides from the 3′ side of the start codon;
(c) providing an expression system for the protein;
(d) introducing the nucleic acid sequences combined in step (b) into the expression system; and
(e) forming the stem-loop structure wherein the length of the stem is in the range of 4-12 nucleotides.

2. The method as claimed in claim 1 further comprising the step of isolating the protein.

3. The method as claimed in claim 1 wherein the heterologous nucleic acid sequence has a length of up to 201 nucleotides.

4. The method as claimed in claim 3 wherein the heterologous nucleic acid sequence has a length of up to 45 nucleotides.

5. The method as claimed in claim 1 wherein the stem-loop structure is formed 12-21 nucleotides from the 3′ side of the start codon.

6. The method as claimed in claim 1 wherein the region of the heterologous nucleic acid sequence that is on the 5′ side of the stem-loop structure does not form a secondary structure with the 5′ untranslated region of the nucleic acid sequence coding for the protein.

7. The method as claimed in claim 1 wherein the region of the heterologous nucleic acid sequence that is on the 5′ side of the stem-loop structure and on the 3′ side of the start codon has a GC content of <less than 50%.

8. The method as claimed in claim 1 wherein an in vitro expression system is used.

9. The method as claimed in claim 8 wherein the in vitro expression system is a prokaryotic in vitro expression system.

10. The method as claimed in claim 9 wherein the prokaryotic in vitro expression system comprises a lysate of Escherichia coli or of Bacillus subtilis.

11. The method as claimed in claim 8 wherein the in vitro expression system is a eukaryotic in vitro expression system.

12. The method as claimed in claim 11 wherein the eukaryotic in vitro expression system comprises a lysate selected from the group consisting of a lysate of mammalian cells, reticulocytes, human tumour cell lines, hamster cell lines, other vertebrate cells, oocytes, eggs of fish, eggs of amphibia, insect cell lines, yeast cells, algal cells, and extracts of plant seedlings.

13. The method as claimed in claim 1 wherein the expression system is a prokaryotic in vivo expression system.

14. (canceled)

15. The method as claimed in claim 13 wherein the prokaryotic expression system comprises an E. coli cell or a Bacillus subtilis cell.

16. The method as claimed in claim 1 wherein the expression system comprises a eukaryotic host cell.

17. The method as claimed in claim 16 wherein the eukaryotic host cell is selected from the group consisting of a yeast cell, an insect cell, an amphibian cell, a fish cell, a bird cell, a mammalian cell, and a vertebrate cell.

18. The method as claimed in claim 16 wherein the expression system is a non-human eukaryotic host organism.

19. The method as claimed in claim 1 wherein the nucleic acid sequence coding for the protein is provided by a method selected from the group consisting of cloning, recombination and amplification.

20. The method as claimed in claim 19 wherein the nucleic acid sequence coding for the protein is provided by a two-step polymerase chain reaction.

21. The method as claimed in claim 1 wherein the nucleic acid sequence coding for the protein or the heterologous nucleic acid sequence comprises a codon adapted, based on codon usage, to the expression system.

22. The method as claimed in claim 1 wherein the heterologous nucleic acid sequence comprises coding sequence for a purification domain or for a proteinase recognition domain.

23. A composition for producing a protein, the composition comprising:
(a) a nucleic acid sequence that is heterologous to the nucleic acid sequence coding for the protein wherein the heterologous nucleic acid sequence is inserted into the protein-coding nucleic acid sequence in the correct reading frame and wherein the heterologous nucleic acid sequence forms a stem-loop structure 6-30 nucleotides from the 3′ side of the translation start codon; and
(b) an expression system for the protein.
